SEQUENCE LISTING (5 sequences)

SEQ ID NO: 1 - DNA, 51 bp, Homo sapiens
  misc_feature (26)..(26): n denotes a or g.
agggacacaaaaagggacatactacntgaattactaatatctaagaaaata

SEQ ID NO: 2 - DNA, 51 bp, Homo sapiens
  misc_feature (26)..(26): n denotes a or t
ggttccttgcgactgctgtgaatttngtgatgcacttggatagtctctgtt

SEQ ID NO: 3 - DNA, 51 bp, Homo sapiens
  misc_feature (26)..(26): n denotes c or t
gtaggcaggtggatctgaaatctcanatagtaccaagacacgtgactagga

SEQ ID NO: 4 - DNA, 51 bp, Homo sapiens
  misc_feature (26)..(26): n denotes c or t
tagcagttcaggtcctaaggcatganattgattaagtgtctgatgagaatt

SEQ ID NO: 5 - DNA, 51 bp, Homo sapiens
  misc_feature (26)..(26): n denotes g or t
gtgtttcagcttggcacacagaaacngttttaatttaacagtccagctcct

(19) United States
(12) Patent Application Publication
(10) Pub. No.: US 2014/0322370 A1
(43) Pub. Date: Oct. 30, 2014

(54) KIT AND METHOD FOR PREVENTING OR TREATING OBESITY

(71) Applicant: Natures Remedies Ltd., Cambridge (DK)

(72) Inventors: Trevor Rodney Jarman, Cambridge (GB); Lasse Hessel, Svendborg (DK)

(73) Assignee: Natures Remedies Ltd., Cambridge (GB)

(21) Appl. No.: 14/326,979

(22) Filed: Jul. 9, 2014

Related U.S. Application Data
(63) Continuation-in-part of application No. 12/824,509, filed on Jun. 28, 2010.
(60) Provisional application No. 61/222,141, filed on Jul. 1, 2009.

(51) Int. Cl. A61K 36/77; A61K 36/185

(52) U.S. Cl. CPC A61K 36/77; A61K 36/185
USPC 424/774

(57) ABSTRACT

A kit and method for preventing or treating obesity are provided, which include a probe for identify a subject at risk of being obese and a composition composed of yerbe maté extract, guarana extract, and damiana extract to decrease the consumption of calories and carbohydrates, increase GLP-1 levels and decrease ghrelin levels.

This application is a continuation-in-part application of U.S. patent application Ser. No. 12/824,509, filed Jun. 28, 2010, which claims benefit of priority to U.S. Provisional Application Ser. No. 61/222,141, filed Jul. 1, 2009, the contents of which are incorporated herein by reference in their entireties.

### BACKGROUND OF THE INVENTION

The necessary condition for the reduction of body mass is a negative energy balance. Energy intake must be consistently lower than energy expenditure in order for weight loss to occur. Therefore, any weight control strategy must address one or both parts of the energy equation; intake or expenditure. With regard to the control of energy intake, two broad strategies can be adopted: the enhancement of the satiety response to food or the blockade of absorption. The physical and chemical properties of various foods can be used to achieve both effects.

With specific regard to the modulation of appetite, those processes responsible for the termination of a meal and the suppression of subsequent intake are of particular interest. The within meal processes of satiation and the post-meal end state of satiety are generated by the sensory, physical and chemical characteristics of the food consumed. The strength of these signals determines meal duration and meal size, and the length of the post-meal interval before the next eating occasion. The activation of such signals can be employed to enhance the appetite response to food and limit caloric intake.

Various naturally occurring ingredients including herbal extracts have been shown to produce beneficial effects on appetite and weight control when used as supplements or food components (Ruxton, et al. (2005) Br. Food J. 107:111-125; Ruxton, et al. (2007) Br. Food J. 109: 416-428). ZOTRIM is a mixed herbal preparation containing Yerbe Maté, Guarana and Damiana, common ingredients of commercially available drinks, and the product is available in the UK as a food supplement. This herb extract formulation significantly delays gastric emptying, reduced the time to perceived gastric fullness and induced significant weight loss over 45 days in overweight patients (Anderson & Fogh (2001) J. Hum. Nutr. Dietet. 14:243-250). A consumer study has also been undertaken to test the efficacy of this preparation in the field. A total of 48 free-living subjects completed a 28-day trial and demonstrated a self-reported mean weight loss of 2.3 kg. Questionnaire data suggested that subjects ate less at meals and snacked less frequently (Ruxton (2004) Nutr. Food Sci. 34:25-28; Ruxton, et al. (2005) Nut. Food Sci. 35:303-331; Ruxton, et al. (2007) supra). However, the effects of ZOTRIM on human food intake, feeding behaviour and subjective feelings of appetite and satiety were not determined.

FIBRESURE is a 100% natural fiber supplement that can be taken daily. The term fiber covers a wide variety of substances belonging to the family of carbohydrates that resist hydrolysis by human alimentary enzymes but are fermented by colonic micro flora (Bianchi & Capurso (2002) Dig. Liver Dis. 34(Suppl 2):S129-33). Fiber is normally connected with increases in satiety due to its high viscosity and bulking effect (Burton-Freeman (2000) J. Nutr. 130:272S-275S). However, FIBRESURE is a fiber product with little effect on viscosity. Current recommendations for the management of obesity and diabetes mellitus include an increase in dietary fiber intake, as it may contribute to lower fasting and postprandial plasma glucose concentrations and improvement of glycaemic control, which can help control energy intake (Vinik & Jenkins (1988) Diabetes Care 11:160-173). FIBRESURE contains the soluble fiber inulin, which is a prebiotic carbohydrate derived from chicory root. Inulin and inulin-type fructans are mostly oligosaccharides or oligofructoses and stimulate colonic production of Short Chain Fatty Acids (SCFAs), (Guarner (2005) Brit. J. Nutr. 93:S61-5). Fiber fermentability which produces SCFA has been linked with increasing satiety (Bosch (2008) Br. J. Nutr. 102:318-325).

The mechanism by which inulin and inulin-type fructans exert a satiating effect has not been identified. There are many gastrointestinal peptides that affect food intake such as ghrelin, cholecystokinin (CCK), glucagon-like peptide-1 (7-36) amide (GLP-1), oxyntomodulin, peptide YY (PYY) and pancreatic polypeptide (PP). Fructans modulate gastrointestinal peptides involved in the control of food intake, particularly GLP-1 and ghrelin (Orskov, et al. (1989) J. Biol. Chem. 264(22):12826-12829). GLP-1 is an anorectic peptide secreted by the L-cells which suppresses meal-induced gastric acid and pancreatic juice secretion and slows gastric emptying (Schjoldager, et al. (1989) Dig. Dis. Sci. 34:703-708). There are several studies showing that peripheral injection of GLP-1 decreases food intake and consequently body weight in rats and human subjects, (Meier, et al. (2002) Eur. J. Pharmacol. 440:269-279; Zander, et al. (2002) Lancet 359:824-830). Ghrelin is associated with the mesolimbic cholinergic dopaminergic reward system. This reward link is composed of cholinergic input from the laterodorsal tegmental area to the mesolimbic dopamine system that originates in the ventral tegmental area and projects to the nucleus accumbens (Jerlhag, et al. (2007) Addict. Biol. 12(1):6-16). In this respect, treatment of human volunteers with approximately 20 g of oligofructose per day for 7 days increased serum GLP-1 levels (Piche, et al. (2003) Gastroenterology 124(4):894-902). Furthermore, in a study of 14 healthy volunteers, it was found that the gut peptide GLP-1 decreases motility in the antro-duodeno-jejunal region thus inducing satiety (Hellstrom, et al. (2008) Neurogastroenterol. Motif. 20(6):649-59).

### SUMMARY OF THE INVENTION

The present invention is a kit containing a probe that specifically hybridizes to an allele of a fat mass and obesity-associated (FTO) gene, which is correlated with obesity in humans; and a composition containing yerba maté leaf extract, guarana seed extract, and damiana leaf extract. In one embodiment, the allele is a single nucleotide polymorphism (SNP) selected from the group of rs9930506, rs9939609, rs1121980, rs1421085, and rs17817449. In another embodiment, the kit further includes a fermentable dietary fiber.

The present invention is also a method for preventing or treating obesity by detecting in a subject the presence of an allele of a FTO gene, which is correlated with obesity in humans, and administering to the subject a composition containing yerba maté leaf extract, guarana seed extract, and damiana leaf extract. In one embodiment, the allele is a SNP selected from the group of rs9930506, rs9939609, rs1121980, rs1421085, and rs17817449. In another embodiment, the method further includes the administration of a fermentable dietary fiber. In a further embodiment, the composition is administered before a meal. In still a further embodiment, the composition modulates hunger hormone levels, e.g., increases GLP-1 levels and decreases ghrelin levels.

### BRIEF DESCRIPTION OF THE DRAWINGS

FIG. 1 depicts the study design.

FIG. 2 shows the ghrelin profile across the study.

FIG. 3 shows the GLP-1 profile across the study.

### DETAILED DESCRIPTION OF THE INVENTION

It has now been found that a combination of selected herbal extracts (i.e., yerbe maté extract, guarana extract, damiana extract) can significantly decrease the consumption of calories and carbohydrates, increase GLP-1 levels and decrease ghrelin levels in subjects with a BMI of between 25 and 39.9 kg/m². As such, this analysis indicates that the combination of herbal extracts can modulate hunger hormone levels and prevent/treat obesity. Accordingly, the present invention features a kit and method for identifying a subject at risk of being obese and providing to the subject a composition composed of yerbe maté extract, guarana extract, and damiana extract.

Fat mass and obesity-associated protein, also known as alpha-ketoglutarate-dependent dioxygenase or FTO, is an enzyme that in humans is encoded by the FTO gene located on chromosome 16. Variants of the FTO gene have been shown to be correlated with obesity in humans (Gerken, et al. (2007) Science 318:1469-72). Accordingly, the present invention features the identification of subjects at risk of being obese by detecting one or more FTO alleles, which have been correlated with obesity in humans. For example, FTO single nucleotide polymorphism (SNP) rs9930506 shows the strongest association with BMI (p=8.6×10⁻⁷), hip circumference (p=3.4×10⁻⁸) and weight (p=9.1×10⁻⁷) (Scuteri, et al. (2007) PLoS Genet. 3:e115). Homozygotes with the rare “G” allele are 1.3 BMI units heavier than homozygotes for the common “A” allele. Similarly, carriers of one copy of the A allele of SNP rs9939609 weigh on average 1.2 kilograms (2.6 lb) more than people with no copies (Frayling, et al. (2007) Science 316:889-94). Carriers of two copies (16% of the subjects) weigh 3 kilograms (6.6 lb) more and have a 1.67-fold higher rate of obesity than those with no copies. SNP rs9939609 in the FTO gene was further confirmed to associate with obesity in two large genome wide association studies of BMI (Thorleifsson, et al. (2009) Nat. Gene. 41:18-24; Willer, et al. (2009) Nat. Genet. 41:25-34). Furthermore, morbid obesity has been shown to be associated with a combination of the rs9939609 allele and INSIG2 single nucleotide polymorphisms (Chu, et al. (2008) Arch. Surg. 143:235-40). In another study, rs9939609 was found in a linkage block in the FTO gene with rs1121980, wherein this block was associated with early onset obesity (Hinney, et al. (2007) PLoS One 2:e1361). In addition, the three FTO SNPs, rs1421085, rs17817449, and rs9939609, are in strong linkage disequilibrium (pairwise r²>0.97), and there are two primary haplotypes, C-G-A (42.0%; this is the haplotype representing obesity risk) and T-T-T (55.5%). Further, rs1121980 has been reported to have the strongest association to early onset obesity compared to several other SNPs all in the same linkage block (Hinney, et al. (2007) PLoS One 2:e1361). Moreover, the effects of variation in two different SNPs in the FTO gene (rs17817449 and rs1421085) have also been studied and the results indicated that there may be an effect on circulating leptin levels and energy expenditure (Do, et al. (2008) Diabetes 57:1147-50). The accumulated data across the multiple, independent studies clearly implicates the FTO gene in humans as having a direct impact on food intake but no effect on energy expenditure.

Accordingly, in certain embodiments, the kit and method of the invention include the detection of an allele of the FTO gene have been shown to be correlated with obesity. In particular embodiments, the allele is one or more of SNP rs9930506, rs9939609, rs1121980, rs1421085, or rs17817449. These SNPs are well-known in the art and can be detected by routine methods. In certain embodiments, a FTO SNP is detected with a probe that specifically hybridizes to the SNP. The term “probe” or “probe sequence” refers to a moiety made of an oligonucleotide or polynucleotide, which contains a sequence complementary to a nucleic acid sequence present in a sample from a subject such that the probe will specifically hybridize under stringent hybridization conditions to the nucleic acid sequence present in the sample under appropriate conditions. The probes of the invention can be associated with a support or substrate to provide an array of nucleic acid probes to be used in an array assay. Moreover, the probe of the invention can be labeled with a detectable label (e.g., a radionuclide, fluorescent dye, or chemiluminescer). The length of the probe of the present invention is generally 5-100 bases, such as 10-100, 10-50, 15-50, 20-50, or 10-40 bases, and preferably, for example, 10-20, 20-30, 30-40, 15-30, 20-40, or 15-25 bases.

The probes of the invention are produced, generated or synthesized according to known methods and specifically hybridize to a target SNP sequence listed in Table 1.

TABLE 1

| | | SEQ ID |
|---|---|---|
| SNP | SNP Sequence* | NO: |
| rs9930506 | AGGGACACAAAAAGGGACATACTAC[A/G] | 1 |
| | TGAATTACTAATATCTAAGAAAATA | |
| rs9939609 | GGTTCCTTGCGACTGCTGTGAATTT[A/T] | 2 |
| | GTGATGCACTTGGATAGTCTCTGTT | |
| rs1121980 | GTAGGCAGGTGGATCTGAAATCTCA[C/T] | 3 |
| | ATAGTACCAAGACACGTGACTAGGA | |
| rs1421085 | TAGCAGTTCAGGTCCTAAGGCATGA[C/T] | 4 |
| | ATTGATTAAGTGTCTGATGAGAATT | |
| rs17817449 | GTGTTTCAGCTTGGCACACAGAAAC[G/T] | 5 |
| | GTTTTAATTTAACAGTCCAGCTCCT | |

*Polymorphism is indicated in brackets as [wild-type/variant].

Probes of the invention can be used in conventional hybridization assays. Generally, such assays involve the hybridization of a probe with a target nucleic acid, followed by detection of the hybrid product in some manner. Hybridization generally occurs under stringent hybridization conditions well-known in the art. Briefly, “stringent hybridization conditions” refers to conditions that are that are compatible to produce duplexes between complementary binding members, i.e., between probes and complementary target nucleic acids in a sample. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution of 50% formamide, 5×SSC (150 mM NaCl, 15 mM trisodium citrate), 50 mM sodium phosphate (pH 7.6), 5×Denhardt's solution, and 10% dextran sulfate, followed by washing the filters in 0.1×SSC at about 65° C.

In some embodiments, the probe is used in conventional hybridization assays such as Southern blot analysis. In other embodiments, the probe is used in the detection of a target nucleic acid using amplification techniques such as polymerase chain reactions. In accordance with such embodiments, the probe is a primer that is capable of acting as a point of initiation of synthesis when placed under conditions in which synthesis of a primer extension product complementary to a nucleic acid strand is induced. Such conditions include the presence of nucleotides (such as the four standard deoxyribonucleoside triphosphates) and an agent for polymerization, e.g., a DNA polymerase, and suitable temperature and pH. The details of such techniques are described, e.g., in U.S. Pat. No. 4,683,195 and U.S. Pat. No. 4,683,202. In this respect, the kit of the invention can also include nucleotides (e.g., A, C, G and T), a DNA polymerase and appropriate buffers, salts and other reagents to facilitate amplification reactions.

In addition to one or more probes that specifically hybridize to one or more alleles of the FTO gene, the kit of the invention also includes a composition for modulating hunger hormone levels and preventing/treating obesity. In this respect, once a subject is identified as being at risk for being obese, as evidenced by the presence of an FTO allele associated with obesity, the subject is provided with a composition to reduce, prevent, or treat obesity. The composition of this invention for preventing/treating obesity is composed of soluble extracts of yerbe maté(leaves of Ilex paraguayensis, I. vomitora, or I. dahoon), guarana (seeds of Paullinia cupana or P. sorbalis) and damiana (leaves of Turnera diffusa var. aphrodisiaca, T. opifera, or T. ulmifoliei). Soluble extracts of the invention can be prepared by conventional methods of drying and/or grinding plant biomass and subjecting the same to one or more suitable solvents, thereby providing an extract, which may be either used as a crude extract or further fractionated.

Suitable methods for drying plant biomass include: sun drying followed by a heated air-drying or freeze-drying; lyophilization or chopping the biomass into small pieces, e.g., 2-10 cm, followed by heated air-drying or freeze-drying. Once sufficient moisture has been removed, e.g., more than 90%, the material can be ground to a coarse particle size, e.g., 0.01-1 mm, using a commercial grinder. For laboratory scale extraction, a coffee grinder or equivalent can be used.

In general terms, a suitable method for preparing an extract of plant biomass includes the steps of treating collected plant biomass with a solvent to extract a fraction having appetite suppressant or curbing activity, separating the extraction solution from the rest of the plant biomass, removing the solvent from the extraction solution and recovering the extract. The extract so recovered may be further purified by way of suitable extraction or purification procedures.

More specifically, plant biomass can be ground to a coarse powder as described above. Subsequently, a suitable solvent, e.g., a food grade solvent, can be added to the powder. A good grade solvent is any solvent which is suitable and approved for use in conjunction with foods intended for human consumption. Examples of suitable solvents are alcohol-based solvents, ethyl acetate, liquid carbon dioxide, hexane, and one or more components of fusel oil, e.g., ethyl acetate. Alcohol-based solvents, i.e., pure alcohol solvents and mixtures thereof with water or other organic solvents, are most desirable.

The extraction solution can then be separated from the residual plant biomass by an appropriate separation procedure such as filtration and/or centrifugation. The solvent can be removed, e.g., by means of a rotary evaporator. The separated crude extract can then be tested to confirm appetite suppressant or appetite curbing activity in a suitable in vivo bioassay.

A suitable and accepted in vivo model for measuring appetite suppression or appetite curbing activity in an animal model is described in Example 3. A clinically effective and medically approved anti-obesity drug, e.g., sibutramine, can be used as a positive control for reduction in food intake in this model. Positive results from this test model are an indicator of clinical efficacy in the human context. Alternatively, suppression, reduction or curbing of appetite can be assessed by any of the methods referred to in WO 98/46243.

Plant extracts of the invention can be dried to remove moisture, e.g., by spray-drying, freeze-drying or vacuum-drying, to yield a free-flowing powder. Optionally, the extracts can be dried on a pharmaceutically acceptable carrier, such as maltodextrin or starch. As yet a further alternative, plant biomass can be extracted and concentrated without drying to give a liquid extract, which is effective in curbing or suppressing appetite.

In addition to yerbe maté, guarana and damiana extract, the composition of the invention can further include one or more of: extracts from the leaves of Buchu (Barosma betulina, B. crenulata, B. serratifolia) containing diosphenol (buchu camphor); extracts from the leaves or flowers of Vervain (Verbena officinales, V. jamaicensis, V. lappulacae, V. hesitate, V. urticifolia, V. Sinuata) containing glycosides, adenosine, essential oils, tannin, livertin and/or emulin; extracts of Kola nut (Cola nitida, C. vera) containing caffeine; or extracts from the leaves or flowers of Ginseng (Panax ginseng, P. quinquefolius L.) containing triterpenoid saponins.

In addition to herbal extracts, the present composition can also include a dietary fiber. As used herein, dietary fiber is the indigestible portion of plant foods that pushes food through the digestive system and absorbs water. Dietary fiber can be soluble (able to dissolve in water) or insoluble (not able to dissolve in water). Soluble fiber, like all fiber, cannot be digested. But it does change as it passes through the digestive tract, being transformed or fermented by bacteria therein. In contrast, insoluble fiber passes through the body largely unchanged. Accordingly, in particular embodiments of the present invention, the dietary fiber is fermentable or soluble.

Fermentable dietary fiber can be obtained from a variety of plant foods, including, but not limited to legumes (e.g., peas, soybeans, and other beans); grains such as oats, rye, chia, and barley; some fruits and fruit juices including prune juice, plums, berries, bananas, and the insides of apples and pears; certain vegetables such as broccoli, carrots and Jerusalem artichokes; root vegetables such as potatoes, sweet potatoes, and onions; and psyllium seed husk. In particular embodiments, the dietary fiber is isolated and/or substantially purified to homogeneity, e.g., at least to 75%, 80%, 85%, or 90%, homogeneity or up to 99% homogeneity.

For use in accordance with the present invention, the isolated, fermentable dietary fiber is inulin, a beta-glucan, a pectin, a natural gum, an oligosaccharide, psyllium seed husk, a resistant dextrin, an alginate or a combination thereof. Inulin, belonging to the class of fibers known as fructans, is typically extracted from enriched plant sources such as chicory roots or Jerusalem artichokes. Beta-glucans, polysaccharides of D-glucose monomers linked by glycosidic bonds, are typically isolated from oat bran, whole oats, oatrim or rolled oats. Pectins, a complex set of polysaccharides that are present in most primary cell walls and particularly abundant in the non-woody parts of terrestrial plants, are composed of a linear chain of α-(1-4)-linked D-galacturonic acid that forms the pectin-backbone. Natural gums, which are polysaccharides of natural origin that are capable of causing a large viscosity increase in solution, include, but are not limited to xanthan gum, acacia gum or guar gum. Alginate is a soluble fiber extracted from seaweed. According to the present invention, oligosaccharides, saccharide polymers containing a small number (typically three to ten) of component sugars (also known as simple sugars), particularly include fructooligosaccharides. As is conventional in the art, a resistant dextrin is a water-soluble dietary fiber obtained by, e.g., subjecting starch to high-temperature heating and enzymatic hydrolysis with α-amylase and glucoamylase. The selection of the dietary fiber to be used in the composition of the present invention can be dependent upon the form of the composition and the manner in which the formulation is administered, e.g., as multiple different formulations or as one formulation containing yerbe maté extract, guarana extract, damiana extract and dietary fiber. In particular embodiments, the dietary fiber of the instant composition is inulin.

According to this invention, the herbal extracts and optional dietary fiber can be provided as a composition prepared as individual formulations (e.g., the composition includes or comprises a formulation containing yerbe matéextract, a formulation containing guarana extract, a formulation containing damiana extract, and an optional formulation containing a dietary fiber), or the composition can be prepared as a combination of formulations (e.g., the composition includes or comprises a formulation containing yerbe maté extract, guarana extract, and damiana extract), or the composition can be prepared as a single unitary formulation (e.g., the composition includes or comprises a formulation containing yerbe maté extract, guarana extract, damiana extract, and optionally dietary fiber). Moreover, when the composition is prepared as individual or a combination of formulations, said formulations can be the same, e.g., all tablets; or different, e.g., a capsule formulation and a liquid formulation. In addition, when taken as individual formulations, said formulations can be taken simultaneously or consecutively, e.g., within minutes of each other.

Soluble plant extracts, dietary fiber or a combination thereof can be admixed by conventional compounding procedures with any conventional pharmaceutical or nutritionally acceptable excipient, diluent or carrier in the preparation of pharmaceuticals, nutraceuticals, nutritional compositions, such as dietary supplements, slimming compositions, medical nutrition or functional foods. Typically, this involves mixing the active ingredients of the invention together with edible pharmaceutically or nutritionally acceptable solid or liquid carriers and/or excipients, e.g., fillers, such as cellulose, lactose, sucrose, mannitol, sorbitol, and calcium phosphates; and binders, such as starch, gelatin, tragacanth, methylcellulose and/or polyvinylpyrrolidone (PVP). Optional additives include lubricants and flow conditioners, e.g., silicic acid, silicon dioxide, talc, stearic acid, magnesium/calcium stearates and polyethylene glycol (PEG) diluents; disintegrating agents, e.g., starch, carboxymethyl starch, cross-linked PVP, agar, alginic acid and alginates, coloring agents, flavoring agents and melting agents. Dyes or pigments may be added to tablets or dragee coatings, for example, for identification purposes or to indicate different doses of active ingredient.

The composition of the invention can optionally include conventional food additives, such as any of emulsifiers, stabilizers, sweeteners, flavorings, coloring agents, preservatives, chelating agents, osmotic agents, buffers or agents for pH adjustment, acidulants, thickeners, texturizers and the like.

Suitable product formulations according to the present invention include sachets, soft gel, powders, syrups, pills, capsules, tablets, liquid drops, sublinguals, patches, suppositories, and liquids. Also contemplated are food and beverage products containing the composition of the present invention, such as solid food products, like bars (e.g., nutritional bars or cereal bars), powdered drinks, dairy products, breakfast cereals, muesli, candies, confectioneries, cookies, biscuits, crackers, chocolate, chewing-gum, desserts and the like; liquid comestibles, like soft drinks, juice, sports drinks, milk drinks, milk-shakes, yogurt drinks or soups, as well as pet treats, pet foods, etc.

The composition of the invention can be provided as a component of a normal meal, e.g., a nutritional or slimming composition, or dietary supplement, in the form of a health drink, a snack or a nutritionally fortified beverage, as well as a pill, a tablet or a softgel, for example. When used as a snack or dietary supplement it will preferably be consumed between or before meals.

Optionally, the composition according to the invention can be nutritionally complete, i.e., may include vitamins, minerals, trace elements as well as nitrogen, carbohydrate and fatty acid sources so that it may be used as the sole source of nutrition supplying essentially all the required daily amounts of vitamins, minerals, carbohydrates, fatty acids, proteins and the like. Accordingly, the composition of the invention may be provided in the form of a nutritionally balanced complete meal, e.g., suited for oral or tube feeding.

In addition to the herbal extracts, the composition of the invention may also include one or more further active ingredients, e.g., capsaicin (red pepper); fatty acids, especially linoleic acid (LA) and conjugated linoleic acid (CLA); glycomacropeptide (GMP); Long Chain Triglyceride (LCT); enterostatin; galactose; glucuronic acid; hydroxycitrate (HCA); citrus; 3-hydroxy butyrate; medium chain tryglycerides (MCTs); D-tagatose; caffeine; potato extract; green tea extract; epigallocatechin gallate, or other catechins; peptide D4; vitamins B, C and/or E; and chromium picolinate. Alternatively, the composition of the invention may be combined with an anti-obesity drug, such as sibutramine, orlistat (XENICAL™), Hoodia extract, and the like.

Daily dosage of a composition of the present invention would usually be single or multiple servings per day, e.g., once or twice daily, for acute or chronic use. However, benefit may be derived from dosing regimens that can include consumption on a daily, weekly or monthly basis or any combination thereof. Administration of compositions of the invention, e.g., treatment, could continue over a period of days, weeks, months or years, in order, for example, to constantly control the weight, or until a healthy or cosmetically beneficial loss of body weight has occurred. Optimally, the composition of the invention is consumed at least once a day on a regular basis, prior to (i.e., pre-prandial administration), or during a meal. Preferably, the composition of the invention is consumed prior to a meal.

The amount and dosage regimen of the composition of the invention to be administered is determined in the light of various relevant factors including the purpose of administration, the age, sex and body weight of an individual subject, i.e., inter alia on the severity of the subject's obesity or overweight. In this respect, the compositions of the invention can be administered under the supervision of a medical specialist, or may be self-administered.

Preferred delivery formats for the composition of the invention, would be as a dietary supplement containing about 50 mg to about 150 mg, or preferably about 100 mg to about 120 mg, dry weight of yerbe maté extract; about 50 mg to about 120 mg, or preferably about 90 mg to 100 mg, of gaurana extract; about 20 mg to about 50 mg, or preferably about 30 mg to about 40 mg, of damiana extract; and optionally about 2 grams to about 10 grams, or preferably about 5 grams to 7 grams, of dietary fiber.

An illustrative example of a formulation of herbal extracts is 27.5% weight yerbe maté extract, 23.2% weight Guarana, 9% weight Damiana extract, and 40.3% weight of dicalcium phosphate, talc, sodium carboxymethylcellulose, magnesium stearate and hydroxypropylmethylcellulose as additional ingredients.

The present invention also features a method for preventing or treating obesity by detecting in a subject the presence of an allele of the FTO gene, which is correlated with obesity in humans, and administering to the subject a composition containing yerba maté leaf extract, guarana seed extract, and damiana leaf extract. The step of detecting in a subject the presence of an allele of the FTO gene, which is correlated with obesity in humans can be carried out using any well-known method for detecting variant alleles (i.e., PCR amplification, Southern blot analysis, etc.). In particular embodiments, this step of the claimed method includes the use of a probe as described herein that detects the presence of a single nucleotide polymorphism selected from the group of rs9930506, rs9939609, rs1121980, rs1421085, and rs17817449.

Upon detecting the presence of an FTO allele associated with the risk of obesity, the subject is administered a composition containing yerba maté leaf extract, guarana seed extract, and damiana leaf extract. Administration of the composition of the present invention results in a 3% to about 11% decrease in calorie intake (Kcal) and a 3% to about 14% decrease in carbohydrate intake (g) compared to a subject not receiving the composition. In particular embodiments, the composition of the invention results in as much as about an 11% decrease in calorie intake (Kcal) and as much as about a 14% decrease in carbohydrate intake (g) when consumed before or with lunch.

Given that the inclusion of a dietary fiber can further decrease food consumption or calorie intake when combined with herbal extracts (see parent application U.S. Ser. No. 12/824,509), such a combination treatment can achieve a 10% to 40% decrease in food consumption (gram weight) or at least a 10% to 35% decrease in calorie intake (Kcal) as compared to a subject not receiving the composition. Such levels clearly surpass other nutritional weight loss compositions. For example, while humans studies analyzing the effects of Caralluma fimbriata have shown a 8.2% reduction in energy intake (Kuriyan, et al. (2007) Appetite 48:338-344), human studies of sodium alginate showed a 7% reduction in energy intake (Paxman, et al. (2008) Appetite 51:713-719), human studies of oligofructose (soluble fermentable non-viscous fiber) have shown a 5% reduction in total intake (Cani, et al. (2006) Euro. J. Clin. Nutr. 60:567-572), and human studies of hydroxycitric acid (HCA-SX) and a combination of HCA-SX and niacin-bound chromium (NBC) and Gymnema sylvestre extract (GSE) have shown a 4% decrease in food intake (Preuss, et al. (2004) Nutr. Res. 24:45-58), human studies with a natural dietary compound of chromium picolinate, inulin, capsicum, L-phenylalanine, and other lipotropic nutrients has not shown any significant difference in energy intake (Hoeger, et al. (1998) Adv. Ther. 15:305-14). Similarly, food intake and appetite ratings were not significantly reduced when either beta-glucan and fructooligosaccharides are used alone or in combination (Peters, et al. (2009) Am. J. Clin. Nutr. 89:58-63; Kim, et al. (2006) Cer. Foods World, pg. 29), or with a fiber system of alginate and guar gum (Mattes (2007) Physiol. Behay. 90:705-711), or with supplements of fermentable fibers (pectin, beta-glucan) and non-fermentable methylcellulose (Howarth, et al. (2003) J. Nutr. 133:3141-3144). Indeed, the levels of reduction in food/calorie intake of the instant composition are more comparable to pharmacological options including sibutramine (12-26% reduction; Rolls, et al. (1998) Obes. Res. 6:1-11; Chapelot, et al. (2000) Physiol. Behay. 68:299-308), diethylpropion (11-15% reduction; Porikos, et al. (1980) Clin. Pharmacol. Ther. 27:815-822), fluoxetin (13-16% reduction; McGuirk & Silverstone (1990) Int. J. Obes. 14:361-72) and fenfluramine/d-fenfluramine (17-22% reduction; Goodall & Silverstone (1988) Appetite 11:215-288. See, also, Halford, et al. (2007) Drugs 67:27-55 and Halford, et al. (2004) Curr. Drug Targets 5:221-40.

Subjects benefiting from the method of the invention include those at risk of being obese (as evidenced by the presence of a FTO allele disclosed herein), as well as those in need of weight loss, e.g. overweight or obese subjects, and subjects controlling food intake so as not to gain weight. In some embodiments, subjects receiving the composition of this invention are average or slightly overweight, i.e., having a BMI of 18.5-29.9 kg/m². In other embodiments, subjects receiving the composition of this invention are overweight, i.e., having a BMI of greater than 29.9 kg/m². In particular embodiments, subjects receiving the composition of this invention have a BMI in the range of 25 to 39.9 kg/m². In still further embodiments, subjects benefiting from the method of the invention are those that consume high fat food, e.g., food containing greater than 8 g of fat per 100 g.

In accordance with the present method, the administration of the composition of the invention modulates hunger hormone levels. In particular, administration of the composition increases GLP-1 levels and decreases ghrelin levels in a subject receiving treatment as compared to a subject not receiving the composition. More particularly, the composition of the invention decreases ghrelin levels by 10 to 35% as compared to a subject not receiving the composition. Given the benefits of decreasing ghrelin levels, another embodiment of this invention features the inclusion of one or more other herbal extracts known to decrease ghrelin levels. For example, Piper betle leaf extract and Dolichos biflorus seed extract (i.e., LI10903F or LOWAT) have been shown to decrease serum ghrelin levels by 20.85% after 8 weeks (Sengupta, et al. (2012) Lipids Health Dis. 11:176). Similarly, Bofu-tsu-shosan has been shown to significantly decrease plasma acylated-ghrelin levels after a single bolus administration (Azushima, et al. (2013) PLoS One 8:e75560). Accordingly, in particular embodiments, the composition of the invention further includes one or more of Piper betle leaf extract, Dolichos biflorus seed extract, or Bofu-tsu-shosan.

The invention is described in greater detail by the following non-limiting examples.

### Example 1

### Materials and Methods

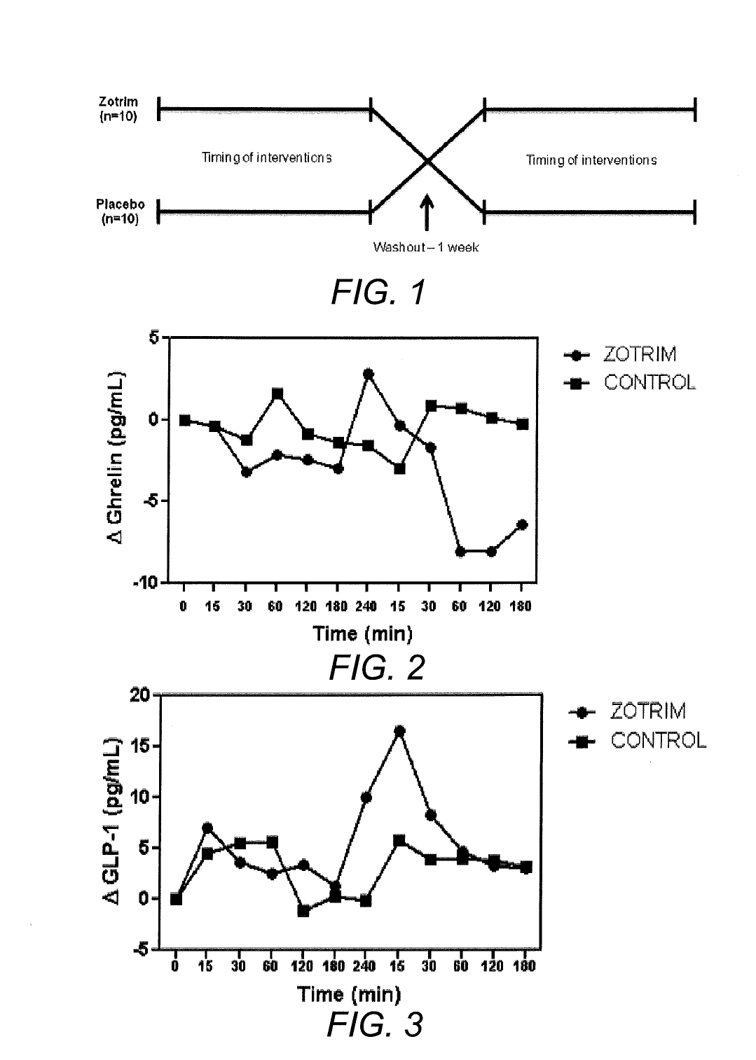

Study Design. A randomized, crossover, placebo controlled and crossover-study was conducted (FIG. 1). The study lasted nine hours with twelve points of blood collection (Table 2). Subjects came to the test site twice followed by a 1-week washout.

TABLE 2

| Time* | Activity |
|---|---|
| 2000 | Fixed-load dinner |
| 0730 | Collect blood sample (+ Glucose) + 3 tablets |
| 0745 | Fixed-load breakfast |
| 0800 | End of breakfast |
| 0815 | Collect blood sample (+Glucose) |
| 0830 | Collect blood sample (+Glucose) |
| 0900 | Collect blood sample (+Glucose) |
| 1000 | Collect blood sample (+Glucose) |
| 1100 | Collect blood sample (+Glucose) |
| 1145 | 3 tablets |
| 1200 | Collect blood sample (+Glucose) + Fixed-load lunch |
| 1215 | End of lunch |
| 1230 | Collect blood sample (+Glucose) |
| 1245 | Collect blood sample (+Glucose) |
| 1315 | Collect blood sample (+Glucose) |
| 1415 | Collect blood sample (+Glucose) |
| 1515 | Collect blood sample (+Glucose) |

*Military time.

Meals.

On the previous day, the subjects had to consume a prescribed dinner at home composed of 585 kcal, 62% carbohydrate, 17% protein, 22% lipid. On day of the study the subjects received a standard fixed-load breakfast containing 495 kcal, 53% carbohydrate, 13% protein, 34% lipid, and a standard fixed-load lunch containing 632 Kcal, 62% carbohydrate, 17% protein and 21% lipid. The food wastage was weighted after meals (FILIZOLA). On the day before, the trial and during the trial, subjects were not allowed to consume alcohol, caffeine or exercise.

Supplement.

The ZOTRIM formulation contained 112 mg Yerbe Mate, 95 mg Guarana and 36 mg Damiana. Guarana, a dough made from the seeds of Paullinia cupana, which grows in Brazil and Venezuela, contains 3-6% caffeine, 5-8.5% tannins, 7.8% resins, 2-3% lipid, 0.06% saponin, 5-6% starch and 1.5% coloring agents (Schery (1954) Plants for Man. London: George Allen and Unwin, pp. 518-519). Yerbe Maté is an extract of Ilex paraguayensis from Brazil, Argentina and Paraguay containing 1-1.5% caffeine, 4-10% tannins and 3% resins and lipids (Hill (1952) Economic Botany. New York: McGraw-Hill Book Company, pp. 479-481.). Damiana is obtained from the leaves of the plant Turnera diffusa var. aphrodisiaca from California, Mexico, Brazil and Bolivia and contains ethereal oils, resins and tannins (Bradley (1992) British Medical Compendium, Vol. 1. London: British Herbal Medical Association, pp. 71-72.).

Blood Collection.

At 7:30 AM, an intravenous catheter was inserted for blood sampling and capillary glycemia was measured by a portable glucometer (ONETOUCH). Serum active ghrelin and GLP-1 concentrations was analyzed by LUMINEX multiplex. At 3:15 PM the catheter was removed.

Subjects.

Ten women with a body mass index (BMI) of between 25 and 39.9 kg/m², aged 20 and 50 years were included in the study. Subjects were excluded from the study if they reported: inclusion in a diet program in the last 12 months; a BMI lower than 25 kg/m² or higher than 40 kg/m²; gastrointestinal symptoms; bariatric surgery; use of medicines that affect appetite or weight; pregnant or planning to become pregnant or breastfeeding; a history of anaphylaxis to food; a general or specific food allergy, including caffeine or lactose and any food offered in the study; immune disease or taking immunosuppressive medication; liver or kidney diseases; chronic alcoholic; taking nutritional supplement or insulin, and endocrine diseases.

Subjects underwent an initial screening including body weight, height, waist circumference, body composition, and completed a questionnaire related to a medical history, smoking behavior, alcohol consumption, eating behavior and physical activity. Body composition was evaluated by dual-energy x-ray absorptiometry (DXA) (Lunar DPX-NT, GE).

Statistical Analysis.

Analysis was performed using Graphpad Prism, Version 5 for WINDOWS. Analysis of variance (ANOVA) was used. The differences between groups in energy intake and macronutrients consumption was analyzed by Student's t-test. Areas under the curve were calculated using the trapezoidal method. Differences were considered significant at p<0.05. Values are expressed as mean±standard deviation.

### Example 2

### Results

The demographic (age), and anthropometric (weight, height, BMI, WC, BLM, BFT) characteristics of the completing participants, together with their screening measure scores are shown in Table 3.

TABLE 3

| | Mean ± SD |
|---|---|
| Variable | n = 10 |
| Age (years) | 34.13 ± 5.91 |
| Weight (kg) | 81.81 ± 7.23 |
| Height (m) | 1.62 ± 0.03 |
| BMI (kg/m²) | 31.34 ± 2.88 |
| Waist Circumference (cm) | 87.56 ± 6.58 |
| Body Lean Mass (%) | 48.68 ± 2.43 |
| Body Fat Mass (%) | 49.55 ± 2.70 |

ZOTRIM group reduced calories and carbohydrate consumption at lunch (P<0.05, Table 4), but did not have a significant effect on glucose profile.

TABLE 4

| | Mean ± SD | |
|---|---|---|
| Meal | Control (n = 10) | ZOTRIM (n = 10) |
| Breakfast (kcal) | 478.30 ± 20.25 | 464.86 ± 22.23 |
| Lunch (kcal) | 567.73 ± 37.39 | 514.64 ± 56.03* |
| Carbohydrate-Breakfast (g) | 61.21 ± 4.88 | 59.75 ± 5.36 |
| Carbohydrate-Lunch (g) | 84.24 ± 7.82 | 72.33 ± 11.58* |
| Protein-Breakfast (g) | 15.71 ± 0.19 | 15.15 ± 0.95 |
| Protein-Lunch (g) | 24.63 ± 1.27 | 23.61 ± 1.86 |
| Lipid-Breakfast (g) | 18.96 ± 0.00 | 18.37 ± 1.04 |
| Lipid-Lunch (g) | 14.69 ± 0.19 | 14.54 ± 0.25 |

*P < 0.05
3-19

ZOTRIM decreased ghrelin levels 30 minutes after breakfast (13%), as well as one (26%) and two (32%) hours after lunch (Table 5). The area under the curve for ghrelin decreased significantly in ZOTRIM group (P<0.05; FIG. 2) and the area under the curve for GLP-1 increased in the same group (P<0.05; FIG. 3).

TABLE 5

| | ZOTRIM | | Control | |
|---|---|---|---|---|
| Time* | Ghrelin | GLP-1 | Ghrelin | GLP-1 |
| 0730 | 32.23 ± 9.86 | 41.67 ± 9.79 | 25.44 ± 7.71 | 48.36 ± 11.96 |
| 0815 | 33.43 ± 14.61 | 47.92 ± 10.95 | 29.33 ± 9.87 | 53.66 ± 9.46 |
| 0830 | 28.05 ± 7.77 | 46.91 ± 11.37 | 23.97 ± 7.91 | 52.33 ± 10.49 |
| 0900 | 31.58 ± 11.79 | 46.44 ± 9.21 | 30.12 ± 7.86 | 53.76 ± 14.16 |
| 1000 | 29.05 ± 9.29 | 47.62 ± 9.40 | 27.49 ± | 49.09 ± 8.76 |
| | | | 10.24 | |
| 1100 | 33.26 ± 13.21 | 49.22 ± 11.92 | 24.13 ± 6.97 | 51.42 ± 8.71 |
| 1200 | 36.46 ± 14.10 | 52.16 ± 10.72 | 27.50 ± 7.88 | 49.19 ± 8.56 |
| 1230 | 35.65 ± 13.13 | 59.10 ± 11.85 | 25.80 ± 6.09 | 56.01 ± 9.04 |
| 1245 | 32.49 ± 9.01 | 52.96 ± 12.96 | 30.17 ± 6.79 | 53.45 ± 9.11 |
| 1315 | 27.12 ± 9.96 | 48.21 ± 12.67 | 29.65 ± 4.96 | 55.26 ± 9.69 |
| 1415 | 24.91 ± 7.83 | 47.51 ± 11.69 | 29.63 ± 5.16 | 52.33 ± 9.73 |
| 1515 | 28.02 ± 8.69 | 51.28 ± 13.89 | 36.81 ± | 53.08 ± 11.52 |
| | | | 11.06 | |

*Military time.

Ghrelin is the only known circulating orexigenic hormone and has been described as the “hunger hormone” with episodic changes in profiles of hunger sensations and levels being similar throughout the day: increasing during fasting and decreasing after food intake. In contrast to ghrelin, GLP-1 is released into the circulation after a meal and is reduced during periods of fasting. GLP-1 is secreted in the distal small and large intestine and therefore is released into the circulation after a meal. GLP-1 is a potent incretin, that is, a stimulator of insulin release. Studies have shown reduced postprandial GLP-1 release in severely obese subjects, which normalizes with weight loss.

In this study, ghrelin was suppressed in the ZOTRIM group. The rise in GLP-1 over the morning in the ZOTRIM group was associated with lower energy intakes at the lunch meal, which is consistent with an influence of GLP-1 on short-term appetite control.

In addition to ghrelin, it is posited that other hormones, which are involved with appetite (e.g., peptide tyrosine tyrosine (PYY), leptin, insulin, GIP and amiline) may be modulated by ZOTRIM such and the analysis of the same will provide a more comprehensive understanding as to how ZOTRIM reduces body fat mass and is of use as an adjuvant in treatment of obesity.

### Example 3

### Animal Model for Food Intake

Studies are conducted with male Sprague-Dawley rats weighing 270-290 grams. Three days before the start of an experiment, the animals are weighed and individually housed. Normal rat chow pellets and tap water are present ad libitum and are provided by food troughs and drinking spouts, which allow continuous recording of the food consumed. Compositions containing plant extracts are administered at predetermined times to the treatment group. Food intake is recorded by continuously weighing the amount of food remaining in a round stainless steel food basket. Food intake is continuously or intermittently recorded over the entire time of an experiment. The weight of each animal is determined on each day of the experiment and recorded, together with any unusual observations, e.g., stressed animals, difficulties with plant extract application, etc. Statistical analysis to detect differences in ingestive behavior between the control group and the treatment group is performed.

What is claimed is:

1. A kit comprising:
(a) a probe that specifically hybridizes to an allele of a fat mass and obesity-associated (FTO) gene, which is correlated with obesity in humans; and
(b) a composition comprising yerba maté leaf extract, guarana seed extract, and damiana leaf extract.

2. The kit of claim 1, wherein the allele comprises a single nucleotide polymorphism selected from the group of rs9930506, rs9939609, rs1121980, rs1421085, and rs17817449.

3. The kit of claim 1, further comprising (c) a fermentable dietary fiber.

4. A method for preventing or treating obesity comprising detecting in a subject the presence of an allele of a fat mass and obesity-associated (FTO) gene, which is correlated with obesity in humans, and administering to the subject a composition comprising yerba maté leaf extract, guarana seed extract, and damiana leaf extract.

5. The method of claim 4, wherein the allele comprises a single nucleotide polymorphism selected from the group of rs9930506, rs9939609, rs1121980, rs1421085, and rs17817449.

6. The method of claim 4, further comprising administering a fermentable dietary fiber.

7. The method of claim 4, wherein the composition is administered before a meal.

8. The method of claim 4, wherein the composition modulates hunger hormone levels.

9. The method of claim 8, wherein the composition increases GLP-1 levels and decreases ghrelin levels.
